(19) United States
(12) Patent Application Publication
(10) Pub. No.: US 2006/0160783 A1
(43) Pub. Date: Jul. 20, 2006

(54) Novel omeprazole forms and related methods

(75) Inventors: Magali Bourghol Hickey, Medford, MA (US); Matthew Peterson, Hopkinton, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(73) Assignee: Transform Pharmaceuticals, Inc., Lexington, MA (US)

(21) Appl. No.: 11/303,503

(22) Filed: Dec. 15, 2005

Related U.S. Application Data
(60) Provisional application No. 60/640,709, filed on Dec. 30, 2004.

(51) Int. Cl. A61K 31/555; A61K 31/4439; C07D 403/02; C07F 3/06

(52) U.S. Cl. 514/185; 514/338; 546/2; 546/273.7

(57) ABSTRACT

The invention provides: (1) novel sodium-containing omeprazole salts formed by the reaction of omeprazole and a sodium source in a crystallization solvent; (2) novel zinc-containing omeprazole salts formed by the reaction of omeprazole and a zinc source in a crystallization solvent, including salts formed by the recrystallization of a zinc salt in a reaction mixture comprising a sodium-containing omeprazole salt and a crystallization solvent; and (3) methods of treatment which use the novel salts to treat or prevent gastric acid-related diseases.

### CROSS REFERENCE TO RELATED APPLICATIONS

The present application claims the benefit of priority of U.S. Provisional Application Ser. No. 60/640,709, filed Dec. 30, 2004, the contents of which are incorporated herein by reference in its entirety.

### FIELD OF THE INVENTION

The invention provides novel forms of omeprazole. These forms include novel sodium and zinc omeprazole salts, co-crystals, hydrates, and solvates. The invention also provides methods of treating or preventing gastric acid-related diseases using the novel forms.

### BACKGROUND OF THE INVENTION

Omeprazole (5-methoxy-2-[[(4-methoxy-3,5-dimethyl-2-pyridinyl)methyl]sulfinyl]-1H-benzimidazole is a substituted benzimidazole that inhibits gastric acid secretion. Omeprazole is a proton pump inhibitor and is useful as an antiulcer agent. Omeprazole may be used for prevention and treatment of gastric-acid related diseases in mammals, and is especially useful in treating gastric acid-related disorders in man. The molecular weight of omeprazole is 345.42 g/mol and the molecular formula of omeprazole is C₁₇H₁₉N₃O₃S. The chemical structure of omeprazole is depicted in FIG. 1 herein.

There are several forms of omeprazole, including those described in U.S. Pat. No. 6,384,059, PCT Publication Nos. WO02/085889 and WO99/00380, and European Patent Office Publications EP0984957 and EP0124495.

Omeprazole has a low water solubility and is chemically unstable in an acidic environment. Further, omeprazole degrades very rapidly in acidic aqueous solutions. While it is only slightly soluble in water, omeprazole is very soluble in alkaline solutions as a negatively charged ion. At pH 6.5, the half-life of degradation of omeprazole is about eighteen hours; at a pH of around 11, the half-life extends to several hundred days. Preformulation studies show that moisture, solvents, and acidic substances have a deleterious effect on the stability of omeprazole.

Specific alkaline salts of omeprazole, including sodium and zinc salts, are disclosed in U.S. Pat. No. 4,738,974 ('974 Patent)(corresponding to EP 124495). According to U.S. Pat. No. 6,207,188 ('188 Patent), the omeprazole sodium salt produced according to examples 1 and 2 of the '974 Patent is a mixture of crystal forms and amorphous material. Further, the '188 Patent discloses that one of the crystal forms present in the mixture of examples 1 and 2 of the '974 Patent (referred to as omeprazole sodium form A) is a hydrate containing one to two water molecules, one of which is bound in the crystal structure and the other of which is easily removed by drying. The resulting dried substance described in examples 1 and 2 of the '974 Patent contains one strongly bound water molecule, is very hygroscopic, and absorbs water rapidly under normal conditions, according to the '188 Patent.

Omeprazole sodium salt form B is disclosed in the '188 Patent. Form B is said to be less hygroscopic than omeprazole form A and is made by treating omeprazole with an aqueous base, Na⁺ B⁻ (where Na denotes sodium and B denotes hydroxide or alkoxide) in an appropriate solvent, such as isopropanol (or isopropanol and water) at ambient temperature.

Example 4 of the '974 Patent discloses di-omeprazole calcium salt dihydrate prepared by dissolving anhydrous CaCl₂ in an aqueous mixture comprising omeprazole sodium salt and thereafter centrifuging and washing the resultant precipitate with deionized water to retrieve the di-omeprazole calcium salt dihydrate.

Despite the fact that there are numerous known forms of omeprazole, the need continues to exist for novel, pharmaceutically-acceptable forms of omeprazole which may exhibit improved pharmacokinetic and metabolic properties and/or which may result in an improved therapeutic profile when administered to patients. Additionally, there is a continuing need for omeprazole forms which are stable over extended periods of time. There is also a particular need for less hygroscopic forms of omeprazole.

### SUMMARY OF THE INVENTION

The invention provides: (1) novel omeprazole sodium salts formed by the reaction of omeprazole and a sodium source in a crystallization solvent; (2) novel omeprazole zinc salts formed by the reaction of omeprazole and a zinc source in a crystallization solvent, including forms formed by the recrystallization of a zinc salt in a reaction mixture comprising a sodium-containing omeprazole form and a crystallization solvent; and (3) methods of treatment which use novel forms of the invention to treat or prevent gastric acid-related diseases.

The invention also provides methods of making novel omeprazole salts (e.g., sodium and zinc), comprising reacting omeprazole or a salt of omeprazole with an appropriate sodium source or zinc source.

Novel omeprazole forms of the invention can be defined by crystallographic parameters, which are described in detail hereinafter. The novel omeprazole forms of the invention are distinguishable from known omeprazole salts—such as the aforementioned sodium salts—on the bases of these crystallographic parameters or other characteristics, such as endothermic or exothermic transitions.

The present invention includes both crystalline and amorphous salts of omeprazole, and any mixtures of both crystalline and amorphous salts of omeprazole.

In certain embodiments, the novel omeprazole forms of the invention are solvates or hydrates. For example, the invention includes a novel sodium omeprazole monohydrate form.

The novel omeprazole forms of the invention: are stable, easy to handle and store, exist in a well-defined state, can be synthesized in a reproducible manner, and should be capable of being manufactured in a full scale production.

### BRIEF DESCRIPTION OF THE FIGURES

FIG. 1 depicts the structural formula for omeprazole.

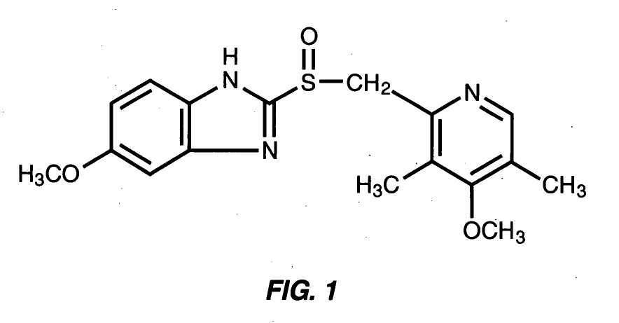

FIG. 2 illustrates differential scanning calorimetry (DSC) measurements of an omeprazole sodium salt.

FIG. 3 illustrates thermogravimetic analysis (TGA) of an omeprazole sodium salt.

FIG. 4 illustrates powder X-ray diffraction (PXRD) measurements of an omeprazole sodium salt.

FIG. 5 illustrates DSC measurements of an omeprazole calcium salt.

FIG. 6 illustrates TGA analysis of an omeprazole calcium salt.

FIG. 7 illustrates PXRD measurements of an omeprazole calcium salt.

FIG. 8 illustrates DSC measurements of an omeprazole zinc salt.

FIG. 9 illustrates TGA analysis of an omeprazole zinc salt.

FIG. 10 illustrates PXRD measurements of an omeprazole zinc salt.

FIG. 11 illustrates an overlay of PXRD measurements of omeprazole sodium salt with and without drying.

### DETAILED DESCRIPTION OF THE INVENTION

As used herein, the following terms have the following respective meanings.

A “solvate” is a complex of variable stoichiometry formed by a solute (either omeprazole or salts, co-crystals, or hydrates of omeprazole) and a liquid at room temperature (about 22 degrees C.), including but not limited to an alcohol (e.g., methanol or ethanol), naphthalene, dioxane, dimethyl sulfoxide, methyl tert-butyl ether, formamide, acetonitrile, nitromethane, methylene chloride, acetic acid, pyridine, 1,4-dioxane, tetrahydrofuran, and 1,2-dichloroethane.

A “polymorph” is a particular crystalline form of an organic compound that exists in a variety of crystal structures. While polymorphic modifications have the same chemical composition, they differ in packing, geometrical arrangement, and other descriptive properties of the crystalline solid state. As such, these modifications may have different solid-state physical properties such as shape, color density, hardness, deformability, stability, and dissolution properties.

“Crystallization solvents” include aromatic hydrocarbons, C₃-C₉ ketones, C₃-C₉ branched alcohols, C₃-C₉ esters, C₅-C₉ hydrocarbons, C₃-C₉ ethers, and cyclic ethers. Aromatic hydrocarbons used as crystallization solvents include C₄-C₆ alkyl aromatic solvents which may include substituted aromatics. Examples of aromatic hydrocarbons include, but are not limited to toluene, benzene, and the like. The term “C₅-C₉ hydrocarbons” refer to C₅-C₉ alkyl solvents which may be substituted, branched or unbranched alkyl. Such hydrocarbon solvents include, but are not limited to straight or branched heptane, octane, pentane, and the like. The term “C₃-C₉ ketones” refers to straight or branched ketones which may optionally be substituted. The term “C₃-C₉ esters” refers to straight or branched esters which may optionally be substituted. The term “ethers” refer to lower alkyl (C₂-C₈) alkyl ethers which may be straight, branched or substituted. The term ether shall include but is not limited to, for example, t-butyl methylether, and the like. The term “cyclic ether” includes C₅-C₇ cyclic ether which may be optionally substituted.

Methylene chloride, for example, is a possible crystallization solvent for use in making sodium-containing omeprazole salts of the invention. Deionized water, for example, is a possible crystallization solvent for use in making zinc-containing omeprazole salts of the invention.

A “sodium source” includes compositions which donate Na⁺, including sodium bases such as sodium hydroxide, sodium carbonate, sodium bicarbonate, or a sodium alkoxide, e.g., sodium methoxide sodium ethoxide, sodium hydride, or sodamide.

A “zinc source” includes compositions which donate Zn⁺⁺, including zinc salts such as zinc chloride and zinc bases such as zinc hydroxide, zinc carbonate and zinc sulphate.

The term “patient” is used throughout the specification to describe an animal, for example a human, to whom treatment, including prophylactic treatment, with the compositions according to the present invention is provided. For treatment of those infections, conditions or disease states which are specific for a given animal such as a human patient, the term patient refers to that animal.

“Gastric acid-related diseases” include but are not limited to gastric ulcer, gastritis, duodenal ulcer, reflux esophagitis, pancreatitis, Zollinger-Ellison syndrome, vacuolating G-cell hyperplasia, basal-mucous-membrane hyperplasia, cholecystitis, attack of biliary colic, dysmotilities of alimentary canal, and irritable bowel syndrome,

The terms “an effective amount”, “therapeutic effective amount”, or “therapeutically effective amount” shall mean an amount or concentration of a composition according to the present invention which is effective within the context of its administration or use, including, for example, the treatment of gastric-acid related diseases.

The invention provides: (1) novel omeprazole sodium salts formed by the reaction of omeprazole and a sodium source in a crystallization solvent; (2) novel omeprazole zinc salts formed by the reaction of omeprazole and a zinc source in a crystallization solvent, including forms formed by the recrystallization of a zinc salt in a reaction mixture comprising a sodium-containing omeprazole form and a crystallization solvent; and (3) methods of treatment which use novel forms of the invention to treat or prevent gastric acid-related diseases.

Novel omeprazole forms of the invention can be defined by crystallographic parameters, which are described in detail hereinafter. The novel omeprazole forms of the invention are distinguishable from known omeprazole salts—such as the aforementioned sodium salts—on the bases of these crystallographic parameters or other characteristics, such as endothermic or exothermic transitions.

The present invention includes both crystalline and amorphous salts of omeprazole, and any mixtures of both crystalline and amorphous salts of omeprazole.

In certain embodiments, the novel omeprazole forms of the invention are solvates or hydrates. For example, the invention includes a novel sodium omeprazole monohydrate form.

The novel omeprazole forms of the invention: are stable, easy to handle and store, exist in a well-defined state, can be synthesized in a reproducible manner, and should be capable of being manufactured in a full scale production.

Pharmaceutical Compositions and Dosage Forms

Pharmaceutical dosage forms of the invention can be administered orally, parenterally, by inhalation spray, topically, rectally, nasally, buccally, vaginally or via an implanted reservoir. Oral and parenteral pharmaceutical compositions and dosage forms are possible dosage forms. For example, the oral dosage form is a solid dosage form, such as a tablet, a caplet, a hard gelatin capsule, a starch capsule, a hydroxypropyl methylcellulose (HPMC) capsule, or a soft elastic gelatin capsule. Other dosage forms include an intradermal dosage form, an intramuscular dosage form, a subcutaneous dosage form, and an intravenous dosage form.

Representative dosage forms, and illustrative types and amounts of excipients, include those described in the '974 Patent. Comparable amounts of the novel salts of the invention can be substituted for the active ingredients described in those '974 Patent formulations.

Pharmaceutical compositions and dosage forms of the invention comprise an active ingredient as disclosed herein, e.g., a form such as a co-crystal or solvate forms of sodium and zinc omeprazole salts of the invention. Pharmaceutical compositions and unit dosage forms of the invention typically also comprise one or more pharmaceutically acceptable excipients or diluents. In one embodiment, the pharmaceutical compositions and unit dosage forms of the invention typically also comprise one or more pharmaceutically acceptable excipients or diluents, wherein at least one of the pharmaceutically acceptable excipients or diluents is an antioxidant.

Pharmaceutical unit dosage forms of this invention are suitable for oral, mucosal (e.g., nasal, sublingual, vaginal, buccal, or rectal), parenteral (e.g., intramuscular, subcutaneous, intravenous, intraarterial, or bolus injection), topical, or transdermal administration to a patient. Examples of dosage forms include, but are not limited to: tablets; caplets; capsules, such as hard gelatin capsules, starch capsules, hydroxypropyl methylcellulose (HPMC) capsules, and soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., non-aqueous liquid suspensions, oil-in-water emulsions, or water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The composition, shape, and type of dosage forms of the invention will typically vary depending on their use. For example, a dosage form used in the acute treatment of a disease or disorder may contain larger amounts of the active ingredient than a dosage form used in the chronic treatment of the same disease or disorder. Similarly, a parenteral dosage form may contain smaller amounts of the active ingredient than an oral dosage form used to treat the same disease or disorder. These and other ways in which specific dosage forms encompassed by this invention will vary from one another will be readily apparent to those skilled in the art. See, e.g., Remington's Pharmaceutical Sciences, 18th ed., Mack Publishing, Easton Pa. (1990) or Remington: The Science and Practice of Pharmacy, 19th ed., Mack Publishing, Easton Pa. (1995).

Typical pharmaceutical compositions and dosage forms comprise one or more excipients. Suitable excipients are well known to those skilled in the art of pharmacy, and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets or capsules may contain excipients not suited for use in parenteral dosage forms. In addition, pharmaceutical compositions or dosage forms may contain one or more compounds that reduce or alter the rate by which the active ingredient will decompose. Such compounds, which are referred to herein as “stabilizers”, include, but are not limited to, antioxidants, pH buffers, or salt buffers.

One or more antioxidants can be used in pharmaceutical compositions and dosage forms to deter radical oxidation of the active ingredient, wherein such antioxidants include, but are not limited to, ascorbic acid, phenolic antioxidants including, but not limited to, butylated hydroxyanisole (BHA) and propyl gallate, and chelators including, but not limited to citrate, EDTA, and DTPA. Optionally, in cases where radical oxidation of the active ingredient is known to occur, a combination of phenolic antioxidants and chelators can be used.

Like the amounts and types of excipients, the amounts and specific type of active ingredient in a dosage form may differ depending on factors such as, but not limited to, the route by which it is to be administered to patients. However, typical dosage forms of the invention comprise a sodium or zinc-containing salt of omeprazole in an amount of from about 10 mg to about 1000 mg, optionally in an amount of from about 25 mg to about 500 mg, or in an amount of from 40 mg to 400 mg, or in an amount of from about 50 mg to about 200 mg.

Oral Dosage Forms

Pharmaceutical compositions of the invention that are suitable for oral administration can be presented as discrete dosage forms, such as, but not limited to, tablets (including without limitation scored or coated tablets), pills, caplets, capsules (including without limitation hard gelatin capsules, starch capsules, HPMC capsules, and soft elastic gelatin capsules), chewable tablets, powder packets, sachets, troches, wafers, aerosol sprays, or liquids, such as but not limited to, syrups, elixirs, solutions or suspensions in a non-aqueous liquid, an oil-in-water emulsion, or a water-in-oil emulsion. Such compositions contain a predetermined amount of the active ingredient, and may be prepared by methods of pharmacy well known to those skilled in the art. See generally, Remington's Pharmaceutical Sciences, 18th ed., Mack Publishing, Easton Pa. (1990) or Remington: The Science and Practice of Pharmacy, 19th ed., Mack Publishing, Easton Pa. (1995).

Typical oral dosage forms of the invention are prepared by combining the active ingredient in an intimate admixture with at least one excipient according to conventional pharmaceutical compounding techniques. Excipients can take a wide variety of forms depending on the form of the composition desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, microcrystalline cellulose, kaolin, diluents, granulating agents, lubricants, binders, stabilizers, and disintegrating agents.

Due to their ease of administration, tablets, caplets, and capsules (such as hard gelatin, HPMC, or starch capsules) represent the most advantageous solid oral dosage unit forms, in which case solid pharmaceutical excipients are used. If desired, tablets or caplets can be coated by standard aqueous or nonaqueous techniques. These dosage forms can be prepared by any of the methods of pharmacy. In general, pharmaceutical compositions and dosage forms are prepared by uniformly and intimately admixing the active ingredient(s) with liquid carriers, finely divided solid carriers, or both, and then shaping the product into the desired presentation if necessary.

For example, a tablet can be prepared by compression or molding. Compressed tablets can be prepared by compressing in a suitable machine the active ingredient(s) in a free-flowing form, such as a powder or granules, optionally mixed with one or more excipients. Molded tablets can be made by molding in a suitable machine a mixture of the powdered compound moistened with an inert liquid diluent.

Examples of excipients that can be used in oral dosage forms of the invention include, but are not limited to, binders, stabilizers, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Suitable forms of microcrystalline cellulose include, but are not limited to, the materials sold as AVICEL-PH-101, AVICEL-PH-103, AVICEL RC-581, and AVICEL-PH-105 (available from FMC Corporation, American Viscose Division, Avicel Sales, Marcus Hook, Pa., U.S.A.), and mixtures thereof. An exemplary suitable binder is a mixture of microcrystalline cellulose and sodium carboxymethyl cellulose sold as AVICEL RC-581. Suitable anhydrous or low moisture excipients or additives include AVICEL-PH-103™ and Starch 1500 LM.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the invention is typically present in from about 50 to about 99 weight percent of the pharmaceutical composition or dosage form.

Disintegrants can be used in the pharmaceutical compositions and dosage forms to provide tablets or caplets that disintegrate when exposed to an aqueous environment. Tablets or caplets that contain too much disintegrant may disintegrate in storage, while those that contain too little may be insufficient for disintegration to occur and may thus alter the rate and extent of release of the active ingredient(s) from the dosage form. Thus, a sufficient amount of disintegrant that is neither too little nor too much to detrimentally alter the release of the active ingredient(s) should be used to form solid oral dosage forms of the invention. The amount of disintegrant used varies based upon the type of formulation and mode of administration, and is readily discernible to those of ordinary skill in the art. Typical pharmaceutical compositions comprise from about 0.5 to about 15 weight percent of disintegrant, for example from about 1 to about 5 weight percent of disintegrant.

Disintegrants that can be used to form pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, clays, other algins, other celluloses, gums, and mixtures thereof.

Antioxidants can be used in the pharmaceutical compositions and dosage forms to deter degradation or radical oxidation of the active ingredient. Examples of suitable antioxidants include, but are not limited to, ascorbic acid, phenolic antioxidants including, but not limited to, butylated hydroxyanisole (BHA) and propyl gallate, and chelators including, but not limited to, citrate, EDTA, and DTPA, or combinations thereof.

Lubricants that can be used to form pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL 200, manufactured by W.R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

Other oral dosage forms for pharmaceutical compositions of the invention are soft elastic gelatin capsules. Soft elastic gelatin capsule unit dosage forms can be made using conventional methods well known in the art. See, e.g., Ebert, Pharm. Tech, 1(5):44-50 (1977). In general, soft elastic gelatin capsules (also known as “soft gels”) have an elastic or soft, globular or oval shaped gelatin shell that is typically a bit thicker than that of hard gelatin capsules, wherein a plasticizing agent, e.g., glycerin, sorbitol, or a similar polyol, is added to a gelatin. The type of gelatin, as well as the amounts of plasticizer and water, can be used to vary the hardness of the capsule shell. The soft gelatin shells may contain a preservative, such as methyl- and propylparabens and sorbic acid, to prevent the growth of fungi. The active ingredient may be dissolved or suspended in a liquid vehicle or carrier, such as vegetable or mineral oils, glycols, such as polyethylene glycol and propylene glycol, triglycerides, surfactants, such as polysorbates, or a combination thereof.

Controlled Release Dosage Forms

Pharmaceutically acceptable salts of omeprazole can be administered by controlled- or delayed-release means. Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. Kim, Cherng-ju, Controlled Release Dosage Form Design, 2 (Technomic Publishing, Lancaster, Pa.: 2000).

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under dosing a drug (i.e., going below the minimum therapeutic levels) as well as exceeding the toxicity level for the drug.

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the omeprazole salts and compositions of the invention. Examples include, but are not limited to, those described in U.S. Pat. Nos.: 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185 B1; each of which is incorporated herein by reference. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), multilayer coatings, microparticles, liposomes, or microspheres or a combination thereof to provide the desired release profile in varying proportions. Additionally, ion exchange materials can be used to prepare immobilized, adsorbed salt forms of omeprazole and thus effect controlled delivery of the drug. Examples of specific anion exchangers include, but are not limited to, Duolite® A568 and Duolite® AP143 (Rohm & Haas, Spring House, Pa. USA).

One embodiment of the invention encompasses a unit dosage form which comprises a pharmaceutically acceptable salt of omeprazole (e.g., a sodium, potassium, or lithium salt), or a polymorph, solvate, hydrate, dihydrate, co-crystal, anhydrous, or amorphous form thereof, and one or more pharmaceutically acceptable excipients or diluents, wherein the pharmaceutical composition or dosage form is formulated for controlled-release. Specific dosage forms utilize an osmotic drug delivery system.

A particular and well-known osmotic drug delivery system is referred to as OROS® (Alza Corporation, Mountain View, Calif. USA). This technology can readily be adapted for the delivery of compounds and compositions of the invention. Various aspects of the technology are disclosed in U.S. Pat. Nos. 6,375,978 B1; 6,368,626 B1; 6,342,249 B1; 6,333,050 B2; 6,287,295 B1; 6,283,953 B1; 6,270,787 B1; 6,245,357 B1; and 6,132,420; each of which is incorporated herein by reference. Specific adaptations of OROS® that can be used to administer compounds and compositions of the invention include, but are not limited to, the OROS® Push-Pull™, Delayed Push-Pull™, Multi-Layer Push-Pull™, and Push-Stick™ Systems, all of which are well known. See, e.g., http://www.alza.com. Additional OROS® systems that can be used for the controlled oral delivery of compounds and compositions of the invention include OROS®-CT and L-OROS®. Id.; see also, Delivery Times, vol. II, issue II (Alza Corporation).

Conventional OROS® oral dosage forms are made by compressing a drug powder (e.g., omeprazole salt) into a hard tablet, coating the tablet with cellulose derivatives to form a semi-permeable membrane, and then drilling an orifice in the coating (e.g., with a laser). Kim, Cherng-ju, Controlled Release Dosage Form Design, 231-238 (Technomic Publishing, Lancaster, Pa.: 2000). The advantage of such dosage forms is that the delivery rate of the drug is not influenced by physiological or experimental conditions. Even a drug with a pH-dependent solubility can be delivered at a constant rate regardless of the pH of the delivery medium. But because these advantages are provided by a build-up of osmotic pressure within the dosage form after administration, conventional OROS® drug delivery systems cannot be used to effectively deliver drugs with low water solubility. Id. at 234.

A specific dosage form of the invention comprises: a wall defining a cavity, the wall having an exit orifice formed or formable therein and at least a portion of the wall being semipermeable; an expandable layer located within the cavity remote from the exit orifice and in fluid communication with the semipermeable portion of the wall; a dry or substantially dry state drug layer located within the cavity adjacent to the exit orifice and in direct or indirect contacting relationship with the expandable layer; and a flow-promoting layer interposed between the inner surface of the wall and at least the external surface of the drug layer located within the cavity, wherein the drug layer comprises a salt of omeprazole, or a polymorph, solvate, hydrate, dihydrate, co-crystal, anhydrous, or amorphous form thereof. See U.S. Pat. No. 6,368,626, the entirety of which is incorporated herein by reference.

Another specific dosage form of the invention comprises: a wall defining a cavity, the wall having an exit orifice formed or formable therein and at least a portion of the wall being semipermeable; an expandable layer located within the cavity remote from the exit orifice and in fluid communication with the semipermeable portion of the wall; a drug layer located within the cavity adjacent the exit orifice and in direct or indirect contacting relationship with the expandable layer; the drug layer comprising a liquid, active agent formulation absorbed in porous particles, the porous particles being adapted to resist compaction forces sufficient to form a compacted drug layer without significant exudation of the liquid, active agent formulation, the dosage form optionally having a placebo layer between the exit orifice and the drug layer, wherein the active agent formulation comprises a salt of omeprazole, or a polymorph, solvate, hydrate, dihydrate, co-crystal, anhydrous, or amorphous form thereof. See U.S. Pat. No. 6,342,249, the entirety of which is incorporated herein by reference.

Topical Dosage Forms

Topical dosage forms of the invention include, but are not limited to, creams, lotions, ointments, gels, shampoos, sprays, aerosols, solutions, emulsions, and other forms know to one of skill in the art. See, e.g., Remington's Pharmaceutical Sciences, 18^{th} ed., Mack Publishing, Easton, Pa. (1990); and Introduction to Pharmaceutical Dosage Forms, 4^{th} ed., Lea & Febiger, Philadelphia, Pa. (1985). For non-sprayable topical dosage forms, viscous to semi-solid or solid forms comprising a carrier or one or more excipients compatible with topical application and having a dynamic viscosity, for example, greater than water, are typically employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure. Other suitable topical dosage forms include sprayable aerosol preparations wherein the active ingredient, for example, in combination with a solid or liquid inert carrier, is packaged in a mixture with a pressurized volatile (e.g., a gaseous propellant, such as freon), or in a squeeze bottle. Moisturizers or humectants can also be added to pharmaceutical compositions and dosage forms if desired. Examples of such additional ingredients are well known in the art. See, e.g., Remington's Pharmaceutical Sciences, 18^{th} ed., Mack Publishing, Easton, Pa. (1990).

Parenteral Dosage Forms

Parenteral dosage forms can be administered to patients by various routes, including, but not limited to, subcutaneous, intravenous (including bolus injection), intramuscular, and intraarterial. Since administration of parenteral dosage forms typically bypasses the patient's natural defenses against contaminants, parenteral dosage forms are optionally sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms of the invention are well known to those skilled in the art. Examples include, without limitation: sterile water; Water for Injection USP; saline solution; glucose solution; aqueous vehicles such as but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate. The solutions can be isotonic and have a physiological pH.

Compounds that increase the solubility the active ingredient(s) disclosed herein can also be incorporated into the parenteral dosage forms of the invention.

Transdermal and Mucosal Dosage Forms

Transdermal and mucosal dosage forms of the invention include, but are not limited to, ophthalmic solutions, patches, sprays, aerosols, creams, lotions, suppositories, ointments, gels, solutions, emulsions, suspensions, or other forms know to one of skill in the art. See, e.g., Remington's Pharmaceutical Sciences, 18^{th} ed., Mack Publishing, Easton, Pa. (1990); and Introduction to Pharmaceutical Dosage Forms, 4^{th} ed., Lea & Febiger, Philadelphia, Pa. (1985). Dosage forms suitable for treating mucosal tissues within the oral cavity can be formulated as mouthwashes, as oral gels, or as buccal patches. Further, transdermal dosage forms include “reservoir type” or “matrix type” patches, which can be applied to the skin and worn for a specific period of time to permit the penetration of a desired amount of active ingredient.

Suitable excipients (e.g., carriers and diluents) and other materials that can be used to provide transdermal and mucosal dosage forms encompassed by this invention are well known to those skilled in the pharmaceutical arts, and depend on the particular tissue or organ to which a given pharmaceutical composition or dosage form will be applied. With that fact in mind, typical excipients include, but are not limited to Labrasol, acetone, ethanol, ethylene glycol, propylene glycol, butane-1,3-diol, isopropyl myristate, isopropyl palmitate, mineral oil, and mixtures thereof, to form dosage forms that are non-toxic and pharmaceutically acceptable.

Depending on the specific tissue to be treated, additional components may be used prior to, in conjunction with, or subsequent to treatment with active ingredients of the invention. For example, penetration enhancers can be used to assist in delivering the active ingredients to or across the tissue. Suitable penetration enhancers include, but are not limited to: acetone; various alcohols such as ethanol, oleyl, an tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as TWEEN 80 (polysorbate 80) and SPAN 60 (sorbitan monostearate).

The pH of a pharmaceutical composition or dosage form, or of the tissue to which the pharmaceutical composition or dosage form is applied, may also be adjusted to improve delivery of the active ingredient(s). Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of the active ingredient(s) so as to improve delivery. In this regard, stearates can serve as a lipid vehicle for the formulation, as an emulsifying agent or surfactant, and as a delivery-enhancing or penetration-enhancing agent. Different hydrates, solvates, salts, or co-crystals of the active ingredient can be used to further adjust the properties of the resulting composition.

In one embodiment of the invention, an active ingredient comprising a sodium or zinc-containing salt of omeprazole is administered orally as needed in an amount of from about 10 mg to about 1000 mg, optionally in an amount of from about 25 mg to about 500 mg, or in an amount from about 40 mg to about 400 mg, or in an amount of from about 50 mg to about 200 mg. The dosage amounts can be administered in single or divided doses. The dosage amounts and frequencies provided above are encompassed by the term “inhibitory effective amount” as used herein.

The suitability of a particular route of administration employed for a particular active ingredient will depend on the active ingredient itself (e.g., whether it can be administered orally without decomposing prior to entering the blood stream) and the disease or disorder to be treated or prevented.

Preparation of Active Ingredient and Forms.

Methods for the preparation of omeprazole are well-known. See European Patent No. 0005129.

A salt of omeprazole may be prepared by reacting omeprazole or a salt of omeprazole with an appropriate sodium source or zinc source such as sodium hydroxide or zinc chloride in accordance with reaction conditions such as those specified in the examples presented hereinafter. For example, the process for forming a salt can be carried out in a crystallization solvent such as methylene chloride in which both reactants (i.e., omeprazole and the sodium or zinc source) are sufficiently soluble.

In one method, in order to achieve crystallization or precipitation, a crystallization solvent is used in which the resulting form, e.g., salt or co-crystal, is only slightly soluble or not soluble. Alternatively, a crystallization solvent is used in which the desired salt is very soluble, and an anti-solvent (or a crystallization solvent in which the resulting salt is poorly soluble) is added to the solution. Other variants for salt formation or crystallization include concentrating the salt and co-crystal solution (e.g., by heating, under reduced pressure if necessary, or by slowly evaporating the solvent, for example, at room temperature), or seeding with the addition of seed crystals, or setting up water activity required for hydrate formation.

In one embodiment, omeprazole or an omeprazole salt is dissolved at atmospheric pressure and at a temperature of between about 10° C. to about 30° C. into an aqueous reaction mixture comprising a sodium or zinc source. The weight ratio of omeprazole to sodium or zinc source in the reaction mixture is approximately 0.5 to approximately 2.0. A crystallization solvent is added to the reaction mixture at a volumetric ratio of approximately 0.1 to 0.5 milliliters of solvent per milliliter of reaction mixture. The resultant aqueous and organic layers are separated, e.g., by extraction. The aqueous layer is washed with polar solvents, (e.g., methylene chloride), concentrated under reduced pressure, and is washed with a polar solvent (for example, an aqueous lower alcohol such as aqueous methanol). The resultant omeprazole crystals are thereafter dried, such as under reduced pressure and heating.

The invention is described further in the following examples, which are illustrative and in no way limiting.

Exemplification

Materials and Methods

Some or all of the following materials and methods were used in the various experiments described in the examples disclosed herein.

### Analytical Equipment and Procedures

Thermogravimetric Analysis

Thermogravimetic analysis of each sample was performed using a Q500 Thermogravimetric Analyzer (TA Instruments, New Castle, Del., U.S.A.), which uses as its control software Advantage for QW-Series, version 1.0.0.78, Thermal Advantage Release 2.0 (©2001 TA Instruments-Water LLC), with the following components: QDdv.exe version 1.0.0.78 build 78.2; RHBASE.DLL version 1.0.0.78 build 78.2; RHCOMM.DLL version 1.0.0.78 build 78.0; RHDLL.DLL version 1.0.0.78 build 78.1; an TGA.DLL version 1.0.0.78 build 78.1. In addition, the analysis software used was Universal Analysis 2000 for Windows 95/95/2000/NT, version 3.1E; Build 3.1.0.40 (©1991-2001 TA Instruments-Water LLC).

For all of the experiments, the basic procedure for performing thermogravimetric analysis comprised transferring an aliquot of a sample into a platinum sample pan (Pan part # 952019.906; (TA Instruments, New Castle, Del. USA)). The pan was placed on the loading platform and was then automatically loaded into the Q500 Thermogravimetric Analyzer using the control software. Thermograms were obtained by individually heating the sample at 10° C./minute across a temperature range (generally from 25° C. to 300° C.) under flowing dry nitrogen (compressed nitrogen, grade 4.8 (BOC Gases, Murray Hill, N.J. USA)), with a sample purge flow rate of 60 mL/minute and a balance purge flow rate of 40 mL/minute. Thermal transitions (e.g., weight changes) were viewed and analyzed using the analysis software provided with the instrument.

Differential Scanning Calorimetry

DSC analysis of each sample was performed using a Q10000 Differential Scanning Calorimeter (TA Instruments, New Castle, Del., U.S.A.), which uses Advantage for QW-Series, version 1.0.0.78, Thermal Advantage Release 2.0 (©2001 TA Instruments-Water LLC), with the following components: QDdv.exe version 1.0.0.78 build 78.2; RHBASE.DLL version 1.0.0.78 build 78.2; RHCOMM.DLL version 1.0.0.78 build 78.0; RHDLL.DLL version 1.0.0.78 build 78.1; an TGA.DLL version 1.0.0.78 build 78.1. In addition, the analysis software used was Universal Analysis 2000 for Windows 95/95/2000/NT, version 3.1E; Build 3.1.0.40 (©2001 TA Instruments-Water LLC).

For all of the DSC analyses, an aliquot of a sample was weighed into an aluminum sample pan (Pan part # 900786.091; lid part # 900779.901 (TA Instruments, New Castle Del. USA)). The sample pan was sealed either by crimping for dry samples or press fitting for wet samples (such as hydrated or solvated samples). The sample pan was loaded into the Q1000 Differential Sanning Calorimeter, which is equipped with an autosampler, and a thermogram was obtained by individually heating the same using the control software at a rate of 10° C./minute from Tₘᵢₙ (typically 30° C.) to Tₘₐₓ (typically 300° C.) using an empty aluminum pan as a reference. Dry nitrogen (compressed nitrogen, grade 4.8 (BOC Gases, Murray Hill, N.J. USA)) was used as a sample purge gas and was set at a flow rate of 50 mL/minute. Thermal transitions were viewed and analyzed using the analysis software provided with the instrument.

Powder X-Ray Diffraction

Powder X-ray diffraction (PXRD) patterns for the samples were obtained using a D/Max Rapid, Contact (Rigaku/MSC, The Woodlands, Tex., U.S.A.), which uses as its control software RINT Rapid Control Software, Rigaku Rapid/XRD, version 1.0.0 (1999 Rigaku Co.). In addition, the analysis software used were RINT Rapid display software, version 1.18 (Rigaku/MSC), and JADE XRD Pattern Processing, versions 5.0 and 6.0 ((1995-2002, Materials Data, Inc.).

For the PXRD analysis, the acquisition parameters were as follows: source was Cu with a K line at 1.5406 Å; x-y stage was manual; collimator size was 0.3 mm; capillary tube (Charles Supper Company, Natick, Mass., U.S.A.) was 0.3 mm ID; reflection mode was used; the power to the X-ray tube was 46 kV; the current to the X-ray tube was 40 mA; the omega-axis was oscillating in a range of 0-5 degrees at a speed of 1 degree/minute; the phi-axis was spinning at an angle of 360 degrees at a speed of 2 degrees/second; 0.3 mm collimator; the collection time was 60 minutes; the temperature was room temperature; and the heater was not used. The sample was presented to the X-ray source in a boron rich glass capillary.

In addition, the analysis parameters were as follows: the integration 2-theta range was 2-60 degrees; the integration chi range was 0-360 degrees; the number of chi segments was 1; the step size used was 0.02; the integration utility was cylint; normalization was used; dark counts were 8; omega offset was 180; and chi and phi offsets were 0.

The relative intensity of peaks in a diffractogram is not necessarily a limitation of the PXRD pattern because peak intensity can vary from sample to sample, e.g., due to crystalline impurities. Further, the angles of each peak can vary by about ±0.1 degrees, preferably ±0.05. The entire pattern or most of the pattern peaks may also shift by about ±0.1 degrees to about ±0.2 degrees due to differences in calibration, settings, and other variations from instrument to instrument and from operator to operator. All reported PXRD peaks in the Figures, Examples, and elsewhere herein are reported with an error of about ±0.1 degrees 2-theta.

For PXRD data herein, including Tables and Figures, each composition of the present invention may be characterized by any one, any two, any three, any four, any five, any six, any seven, or any eight or more of the 2-theta angle peaks. Any one, two, three, four, five, or six DSC transitions can also be used to characterize the compositions of the present invention. The different combinations of the PXRD peaks and the DSC transitions can also be used to characterize the compositions. TGA data can also be used to characterize the compositions of the present invention.

### EXAMPLE 1

Omeprazole Sodium Salt

Sodium hydroxide (116 mg, 2.90 mmol) was dissolved in water (25 mL) and stirred at room temperature for 5 minutes. Omeprazole (1000 mg, 2.90 mmol) was then added to the colorless solution and stirring was continued at room temperature for an additional 5 minutes. Methylene chloride (5 mL) was added to the yellow solution and stirred for 1 minute. The aqueous and organic layers were then separated and the aqueous layer was washed twice with methylene chloride (2×5 mL). The aqueous layer was then concentrated under reduced pressure until a majority of the water was removed and methanol (10 mL) was added to the product.

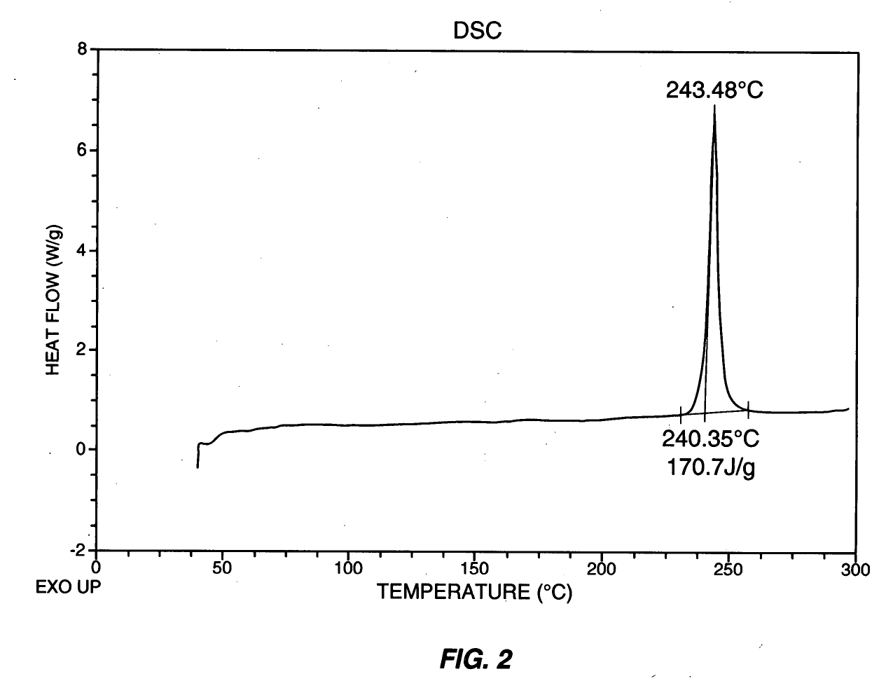

Further drying gave a slightly yellow solid to which ethyl acetate (20 mL) was added and heated at reflux for 20 minutes. After heating the slurry was cooled to room temperature and filtered. The filtered material was washed with diethyl ether and dried to give a white solid (741 mg, 60% TY). DSC, TGA and PXRD patterns are shown in FIGS. 2, 3 and 4, respectively.

DSC of the omeprazole sodium salt characterized in FIG. 2 showed an exothermic transition at about 240 degrees C.

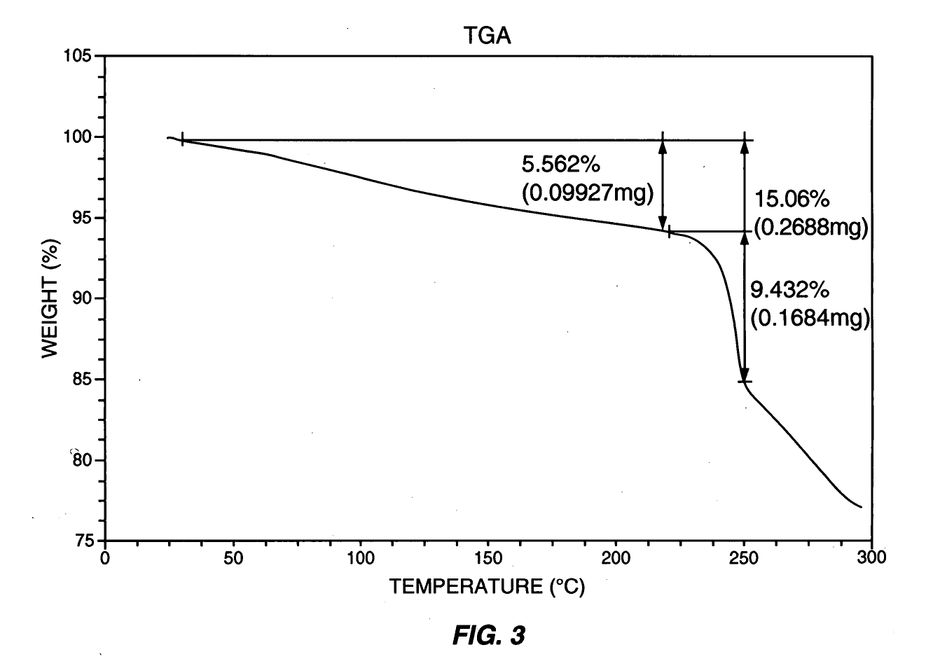

TGA of the omeprazole sodium salt characterized in FIG. 3 showed about a 5.6 percent weight loss between about 30 and about 220 degrees C.

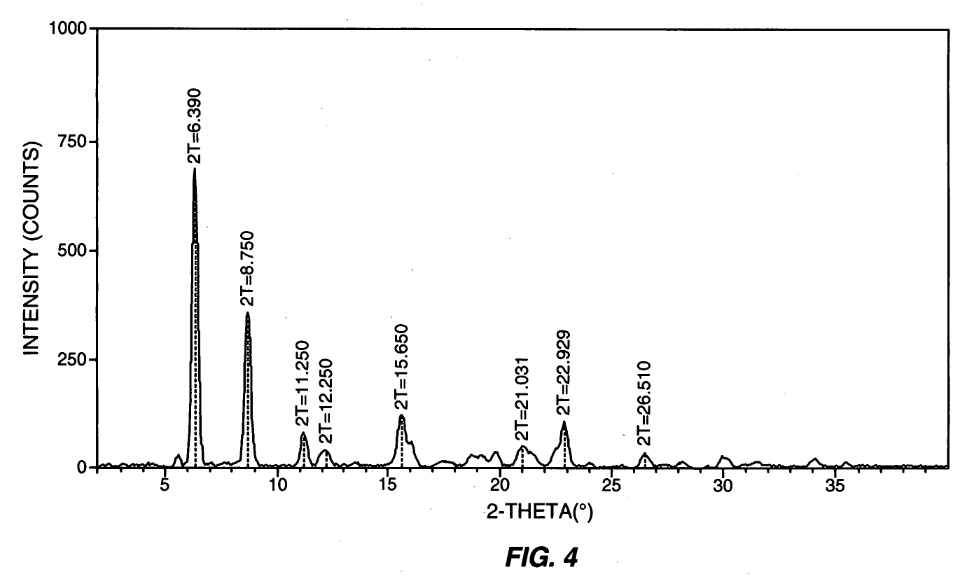

Omeprazole sodium salt can be characterized by any one, any two, any three, any four, any five, or any six or more of the peaks in FIG. 4 including, but not limited to, 6.39, 8.75, 11.25, 12.25, 15.65, 21.03, 22.93 and 26.51 degrees 2-theta (background subtracted).

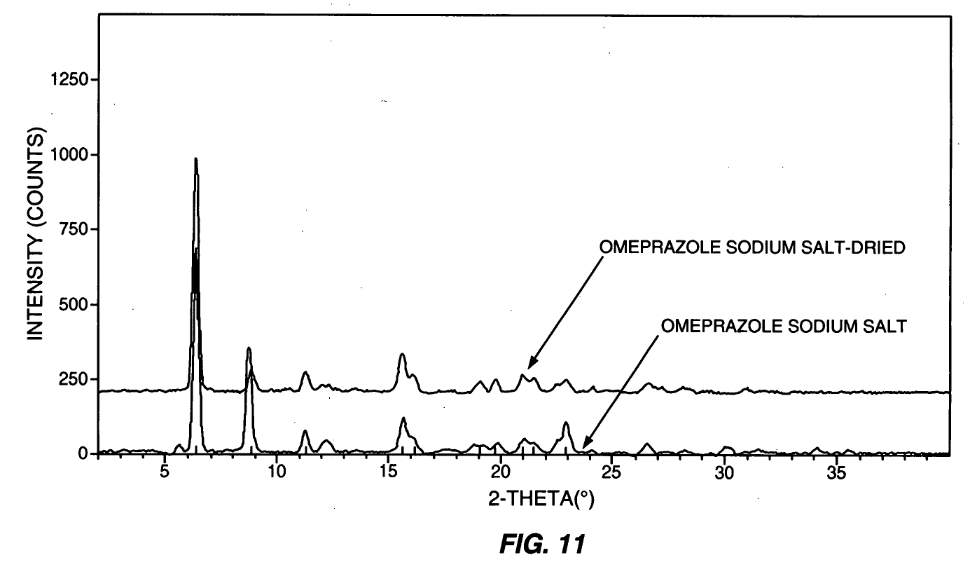

The omeprazole sodium salt obtained according to the method above appears to be a hydrated salt based on the TGA data. A weight loss of about 4.7% would indicate that the salt is a monohydrate. Considering the TGA thermogram, a weight loss of about 5.6% is observed from about 30 degrees C. to about 220 degrees C. prior to decomposition of the salt. It is possible that the salt contained some residual water on the surface that gradually came off with heating. It is important to note that with further drying of omeprazole sodium salt, the peak at 2θ=8.75 degrees begins to decrease in intensity, as shown in FIG. 11.

### EXAMPLE 2

Omeprazole Zinc Salt

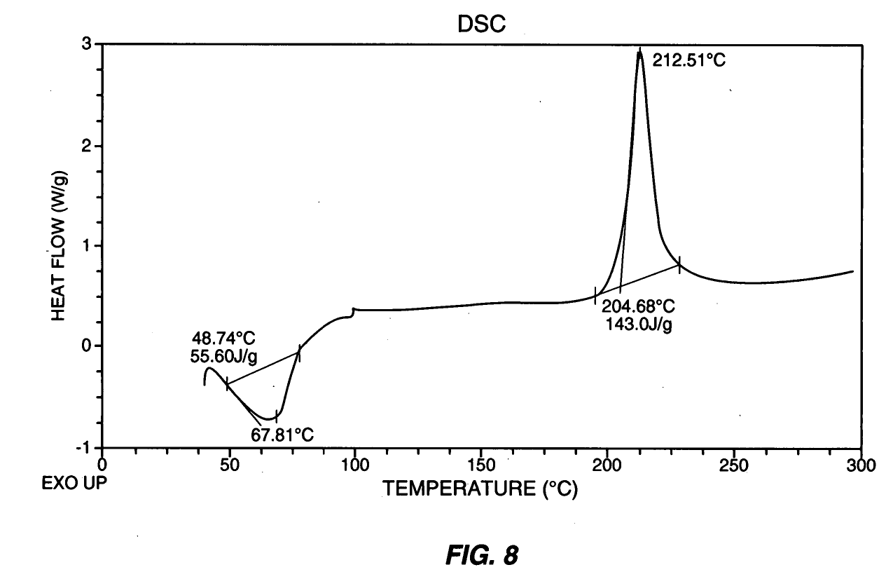

Zinc chloride (76 mg, 0.557 mmol) was dissolved in deionized water (2 mL) and was added drop-wise to a vigorously stirred solution of omeprazole sodium salt (410 mg, 1.113 mmol) from Example 1 dissolved in deionized water (25 mL). The resulting slurry was stirred at room temperature for 1 hour and concentrated under reduced pressure to a total volume of approximately 10 mL. The filtrate was then collected using a Hirsh funnel and washed with a minimal amount of water. The filtered cake was left to dry overnight to give an amorphous colorless solid (318 mg, 75.4% TY). DSC, TGA and PXRD patterns are shown in FIGS. 8, 9 and 10, respectively.

DSC of the omeprazole zinc salt characterized in FIG. 8 showed an endothermic transitions at about 49 degrees C. and an exothermic transition at about 205 degrees C.

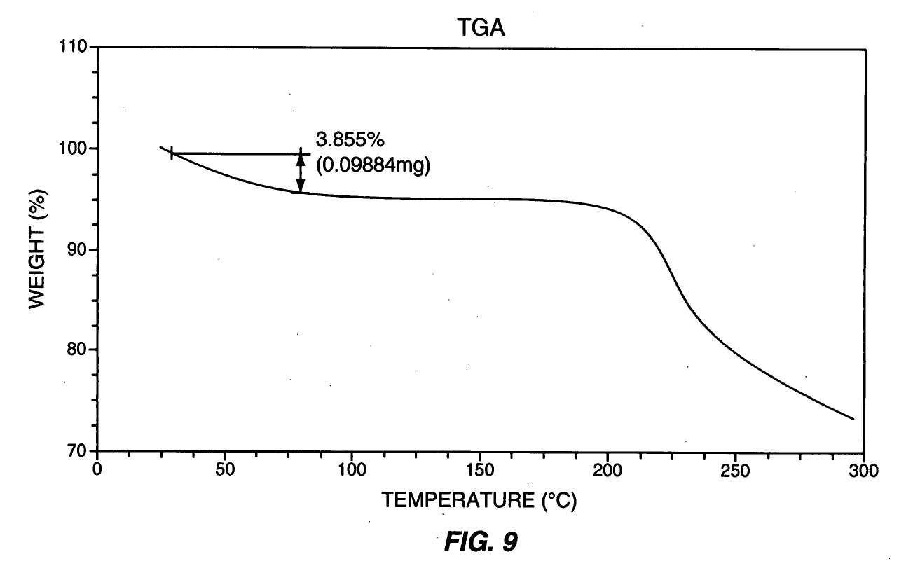

TGA of the omeprazole zinc salt characterized in FIG. 9 showed about a 3.9 percent weight loss between about 30 and about 80 degrees C.

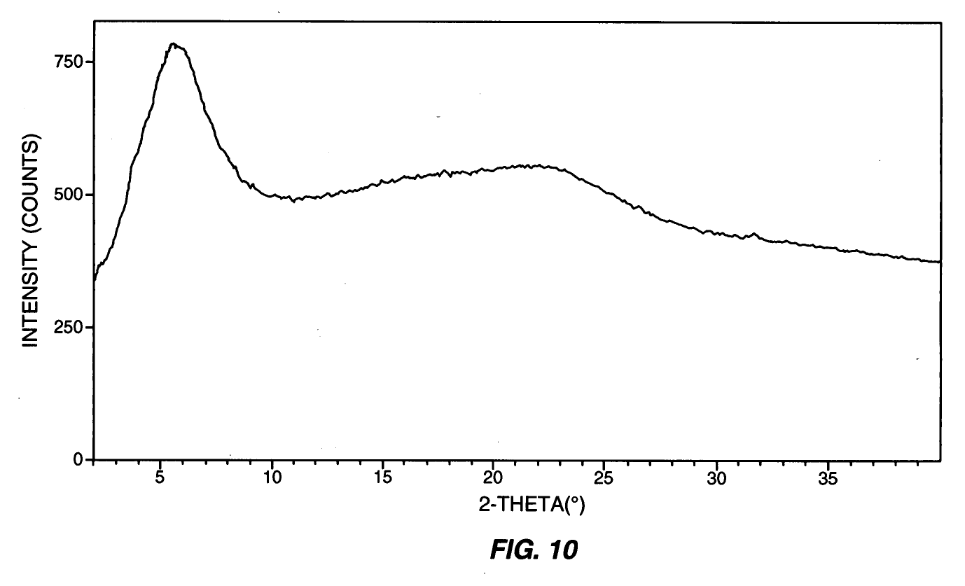

Omeprazole zinc salt can be characterized by any one or more of the peaks in FIG. 10 (background subtracted).

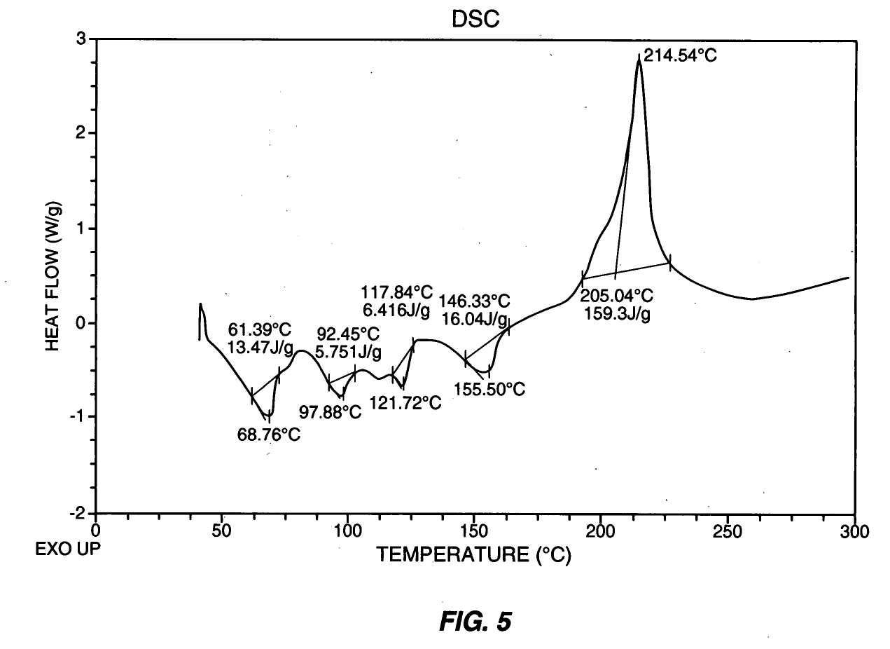

Omeprazole calcium salt was also synthesized in order to attempt to seed the amorphous omeprazole zinc salt with a crystalline divalent omeprazole salt. Calcium chloride (17.9 mg, 0.161 mmol) was dissolved in deionized water (2 mL). The solution was added drop-wise to a vigorously stirred solution of omeprazole sodium salt (125 mg, 0.340 mmol) dissolved in deionized water (12 mL). The resulting slurry wad stirred at room temperature for one hour and was left standing overnight. The precipitate was collected, washed with acetone and dried to give a colorless solid (88 mg, 74.8% TY). DSC, TGA and PXRD patterns are shown in FIGS. 5, 6 and 7, respectively.

DSC of the omeprazole calcium salt characterized in FIG. 5 showed several endothermic transitions at about 61, 92, 118, and 146 degrees C., and an exothermic transition at about 205 degrees C.

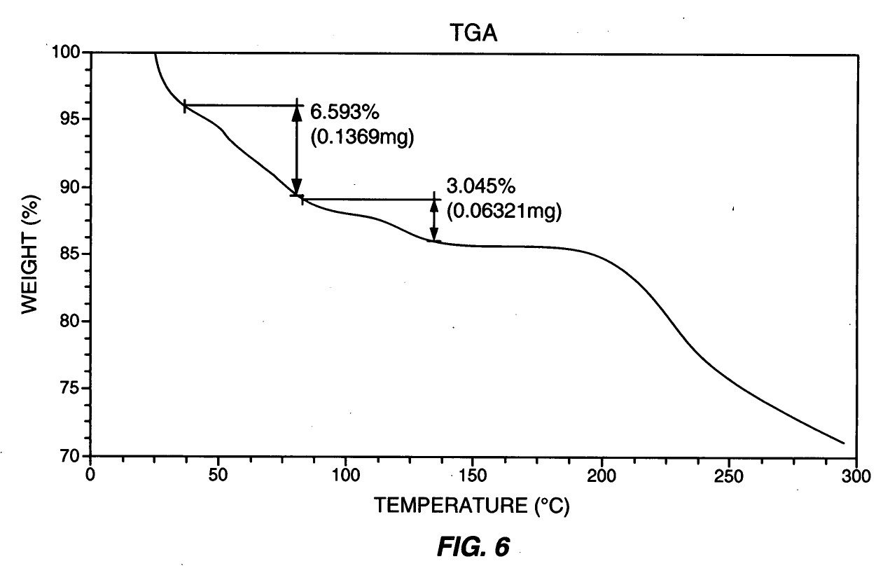

TGA of the omeprazole calcium salt characterized in FIG. 6 showed about a 6.6 percent weight loss and about a 3 percent weight loss between about 30 and about 140 degrees C.

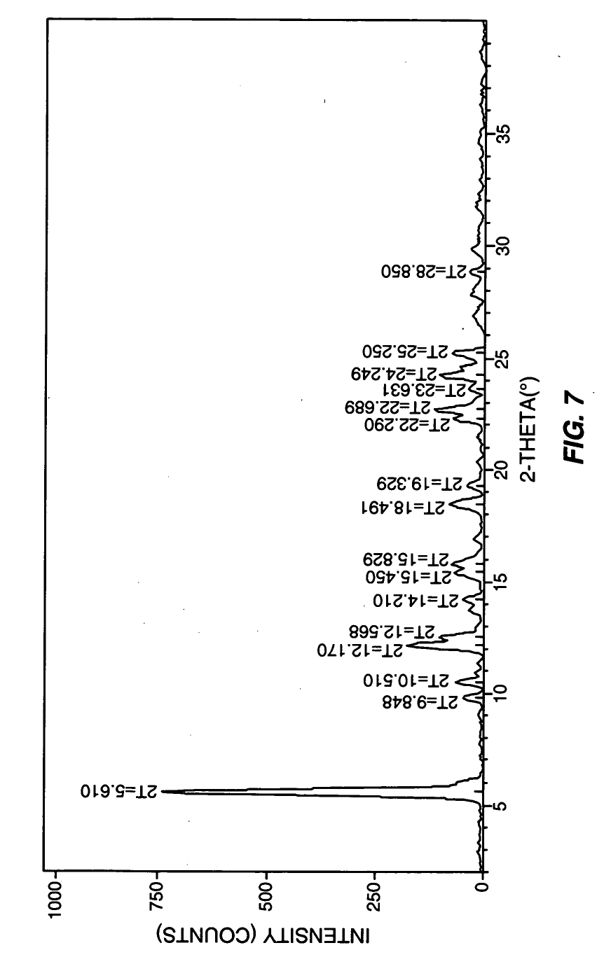

Omeprazole calcium salt can be characterized by any one, any two, any three, any four, any five, or any six or more of the peaks in FIG. 7 including, but not limited to, 5.61, 9.85, 10.51, 12.17, 18.49, 22.29, 22.69, 24.25, and 25.25 degrees 2-theta (background subtracted).

The omeprazole calcium salt, described above, was used in an attempt to seed or induce crystallization of the omeprazole zinc salt. This was completed by adding a small amount of the omeprazole calcium salt with a spatula to the slurry described above in the method of making omeprazole zinc salt. The seeding did not result in a crystalline omeprazole zinc salt.

What is claimed is:

1. An omeprazole sodium salt formed by the reaction of omeprazole and sodium hydroxide, wherein methylene chloride is used as a crystallization solvent.

2. The omeprazole sodium salt of claim 1, wherein the salt is a monohydrate.

3. An omeprazole sodium salt, wherein said salt is characterized by a powder X-ray diffraction pattern comprising peaks expressed in terms of 2-theta angles, wherein:
(a) said X-ray diffraction pattern comprises peaks at 6.39, 8.75, and 15.65 degrees;
(b) said X-ray diffraction pattern comprises peaks at 6.39, 11.25, and 22.93 degrees;
(c) said X-ray diffraction pattern comprises peaks at 21.03, 22.93, and 26.51 degrees; or (d) said X-ray diffraction pattern comprises peaks at 6.39, 8.75, 11.25, 15.65, and 22.93 degrees.

4. The omeprazole sodium salt of claim 3, wherein said salt exhibits:
(a) a PXRD diffractogram substantially as shown in FIG. 4;
(b) a DSC thermogram substantially as shown in FIG. 2; or
(c) a DSC thermogram with an exothermic transition at about 240 degrees C.

5. An omeprazole zinc salt formed by the reaction of omeprazole and a zinc source in a crystallization solvent.

6. The omeprazole zinc salt of claim 5, wherein the crystallization solvent is an aqueous solvent comprising a sodium-containing omeprazole salt and the zinc-containing omeprazole salt is recrystallized in the aqueous solvent.

7. A pharmaceutical dosage form comprising a pharmaceutically acceptable carrier and a therapeutically effective amount of a salt of claim 3.

8. A pharmaceutical dosage form comprising a pharmaceutically acceptable carrier and a therapeutically effective amount of a salt of claim 5.

9. A method of treatment comprising administering a therapeutically effective amount of a salt of claim 3 to a patient suffering from a gastric-acid related disease.

10. A method of treatment comprising administering a therapeutically effective amount of a salt of claim 3 to a patient to prevent the onset of a gastric-acid related disease.

11. A method of treatment comprising administering a therapeutically effective amount of a salt of claim 5 to a patient suffering from a gastric-acid related disease.

12. A method of treatment comprising administering a therapeutically effective amount of a salt of claim 5 to a patient to prevent the onset of a gastric-acid related disease.
